(19) United States
(12) Patent Application Publication
(10) Pub. No.: US 2017/0135679 A1
(43) Pub. Date: May 18, 2017

(54) ENDOSCOPIC BIOPSY INSTRUMENT

(71) Applicants: Charles Walther, Lund (SE); Stephan Dymling, Limhamn (SE); Bruno Walther, Löberöd (SE)

(72) Inventors: Charles Walther, Lund (SE); Stephan Dymling, Limhamn (SE); Bruno Walther, Löberöd (SE)

(21) Appl. No.: 15/317,573

(22) PCT Filed: Jun. 2, 2015
(86) PCT No.: PCT/EP2015/062210

§ 371 Date: Dec. 9, 2016

(30) Foreign Application Priority Data
Jun. 10, 2014 (SE) 1450711-5

(51) Int. Cl. A61B 10/02; A61B 1/267; A61B 10/04

(52) U.S. Cl. CPC A61B 10/0266; A61B 10/04; A61B 1/2676

(57) ABSTRACT

An endoscopic biopsy instrument for obtaining a tissue sample from a body of a patient. The endoscopic biopsy instrument comprising a proximal portion (P) adapted to remain outside of the body of the patient in use, and a distal portion (D) adapted to be inserted into the body of the patient in use. The endoscopic biopsy instrument further comprising a sleeve (9) and a motion transferring member (3). The distal end (3E) of the motion transferring member being adapted to be connected to a rotatable cutting tip (5) being extendable from a distal end of the sleeve, and being configured to obtain a tissue sample from within the body of the patient using rotary motion. The endoscopic biopsy instrument further comprises a rotary motion element (1) for transferring rotary motion to the rotatable cutting tip, a linear motion element (1) for extending and retracting the cutting tip from the distal portion of the sleeve, and a lockable adjustment device (6, 7, 9A) for, in situ, adjusting and locking a maximum length that the rotatable cutting tip extends from the distal portion of the sleeve, when being extended and retracted by the linear motion element.

### TECHNICAL FIELD

The present invention relates to the field of endoscopic biopsy.

### BACKGROUND

A biopsy is a medical test commonly performed by a surgeon involving sampling of cells or tissues for examination. A large variety of endoscopic biopsy instruments are commercially available today, the majority of which are biopsy forceps that operate by the forceps being closed to pinch of the tissue sample.

For some diagnostic purposes, the millimeter-sized samples retrievable using forceps are sufficient, but for some types of lesions and tumors, such a small and superficial sample is inadequate for making a diagnosis. An endoscopic biopsy instrument for obtaining a deeper tissue sample from a patient is for example disclosed in PCT publication WO2014020150 (to Walther).

When taking a tissue sample with an endoscopic biopsy instrument, the instrument is inserted in a working channel of an endoscope, and the forceps are advanced to the biopsy site. The forceps are usually operated by means of a syringe-type handle, which usually offers sufficient precision for operating a forceps.

However, in many applications there is a need for obtaining tissue at a more specific location and at a specific depth. It would therefore be advantageous to have an improved way of controlling the location and depth of the position in which a tissue sample is obtained.

### SUMMARY

An endoscopic biopsy instrument for obtaining a tissue sample from a body of a patient is provided, The endoscopic biopsy instrument comprises a proximal portion adapted to remain outside of the body of the patient in use, and a distal portion adapted to be inserted into the body of the patient in use. The endoscopic biopsy instrument further comprises a sleeve extending from the proximal portion to the distal portion, and a motion transferring member extending from the proximal portion to the distal portion within the sleeve. The motion transferring member is configured to transfer rotary and linear motion.

The distal portion of the endoscopic biopsy instrument comprises a distal end of the motion transferring member, adapted to be connected to a rotatable cutting tip being extendable from a distal end of the sleeve. The rotatable cutting tip is configured to obtain a tissue sample from within the body of the patient using rotary motion.

The proximal portion of the endoscopic biopsy instrument comprises a handle portion connected to the sleeve. The handle portion comprises a rotary motion element connected to the motion transferring member for transferring rotary motion to the rotatable cutting tip, and a linear motion element connected to the cutting tip via the motion transferring member, for extending and retracting the cutting tip from the distal portion of the sleeve. The proximal portion further comprises a lockable adjustment device for, in situ, adjusting and locking a maximum length that the rotatable cutting tip extends from the distal portion of the sleeve, when being extended and retracted by the linear motion element. The lockable adjustment device enables the depth of the tissue sample to be obtained to be controlled, in situ, which increases the precision of the depth control.

According to one embodiment, the linear motion element has an innermost position, in which the cutting tip extends maximally from the distal end of the sleeve. The lockable adjustment device may be adapted to adjust the distance between a proximal end of the sleeve and the innermost position, for adjusting the maximum length that the rotatable cutting tip extends from the distal portion of the sleeve when being extended and retracted by the linear motion element.

According to one embodiment, the lockable adjustment device comprises an adjustment sleeve being axially displaceable in relation to the sleeve, and the lockable adjustment device further comprises a locking device for axially locking the adjustment sleeve in relation to the sleeve and thereby adjusting and locking the maximum length that the rotatable cutting tip extends from the sleeve. In one embodiment, a proximal portion of the sleeve comprises a threaded portion, and the adjustment sleeve comprises threads being adapted to engage the threads of the sleeve, such that the adjustment sleeve can be axially displaced in relation to the sleeve by the adjustment sleeve and the sleeve being radially rotated in relation to each other. The locking device may comprise a threaded locking member adapted to engage the threads of at least one of the sleeve and the adjustment sleeve, for locking the sleeve in relation to the adjustment sleeve. The embodiments with threads enable precise adjustment of the adjustment sleeve and locking member, whilst making sure that the adjustable locking member can withstand exerted forces.

In an alternative embodiment, the lockable adjustment device comprises a locking device comprising a radially displaceable part adapted to engage at least one of the motion transferring member, the sleeve and the adjustment sleeve. The radially displaceable part is thereby adapted to lock the maximum length that the rotatable cutting tip extends from the sleeve, when being extended and retracted by the linear motion element. The adjustment sleeve may be a compressible adjustment sleeve comprising the radially displaceable part, such that compression of the compressible adjustment sleeve disengages the radially displaceable part from the motion transferring member, the sleeve or the adjustment sleeve, such that the maximum length that the rotatable cutting tip extends from the distal portion of the sleeve can be adjusted. The release of the compression of the compressible adjustment sleeve engages the radially displaceable part to the motion transferring member the sleeve or the adjustment sleeve, such that the lockable adjustment device is locked. The radially displaceable part or the compressible adjustment sleeve may comprise a flexible or elastic polymer material.

The rotary motion element and the linear motion element may be a combined element, such that rotary and linear motion can be generated single-handedly.

The endoscopic biopsy instrument according to any one of the preceding embodiments may be configured for stepless operation of at least one of: the adjustment of the maximum length that the rotatable cutting tip extends from the sleeve, the extension and retraction of the rotatable cutting tip, and the rotary motion of the cutting tip.

According to one embodiment the sleeve is a flexible sleeve and/or the motion transferring member is a flexible motion transferring member, such that the endoscopic biopsy instrument can be inserted through a flexible endoscope.

The rotatable cutting tip may comprise a drill, and the flexible motion transferring member may be a flexible shaft.

According to one embodiment, the endoscopic biopsy instrument or the motion transferring member may have a length exceeding one of 10 cm, 20 cm, 30 cm, 40 cm, 50 cm, 60 cm, 70 cm, 80 cm, 90 cm or 100 cm e.g. for being able to reach areas of the patient's digestive system or airways.

The flexible sleeve may comprise a helically wound metal sleeve and/or a flexible sleeve made from a polymer material, and/or a woven or braided flexible metal sleeve.

According to one embodiment, the linear motion element extends partially inside of the adjustment sleeve and the adjustment sleeve extends partially inside of the sleeve, creating a stable elongated handle portion.

The endoscopic biopsy instrument according to any one of the preceding embodiments may comprise grip improving protrusions or recesses on at least one of: the rotary motion element, the linear motion element and the lockable adjustment device. The grip improving protrusions or recesses may be axially extending grooves.

A method of obtaining a tissue sample from a body of a patient is further provided. The method uses an endoscopic biopsy instrument comprising a sleeve and a rotatable cutting tip operated from a handle portion of the endoscopic biopsy instrument. The cutting tip is configured to obtain a tissue sample from within the body of the patient using rotary motion. The method comprises inserting the endoscopic biopsy instrument through an endoscope into the body of the patient, and adjusting a maximum length that the rotatable cutting tip extends from the sleeve, when being extended and retracted by a linear motion element of the handle portion. The method further comprises using a lockable adjustment device of the endoscopic biopsy instrument for locking the lockable adjustment device, and rotating, by means of the handle portion, the rotatable cutting tip for obtaining a tissue sample. After the tissue sample has been obtained, the rotatable cutting tip is retracted into the sleeve using the linear motion element, and the endoscopic biopsy instrument is retracted from the body of the patient.

The method may be preceded by the steps of placing the endoscope such that it extends from a location outside the body of the patient to a location inside the body of the patient, and the step of inflating an area of the patient with a gas for creating a cavity within the body of the patient allowing visual inspection.

The endoscope may comprise a camera for visual inspection, such that the step of adjusting a maximum length that the rotatable cutting tip extends from the sleeve can be performed under visual inspection.

The endoscope may be a gastroscope, and the step of inserting the endoscopic biopsy instrument may comprise inserting the endoscopic biopsy instrument through the gastroscope into the digestive system of the patient, and the step of obtaining a tissue sample may comprise obtaining a tissue sample from the digestive system of the patient.

Alternatively, the endoscope may be a colonoscope, and the step of inserting the endoscopic biopsy instrument through an endoscope into the body of the patient may comprise inserting the endoscopic biopsy instrument through the colonoscope into the digestive system of the patient, and the step of obtaining a tissue sample may comprise obtaining a tissue sample from the digestive system of the patient.

Alternatively, the endoscope may be a bronchoscope, and the step of inserting the endoscopic biopsy instrument through an endoscope into the body of the patient may comprise inserting the endoscopic biopsy instrument through the bronchoscope into the airways of the patient, and the step of obtaining a tissue sample may comprise obtaining a tissue sample from the airways of the patient.

Alternatively, the endoscope may be a rhinoscope, and the step of inserting the endoscopic biopsy instrument through an endoscope into the body of the patient may comprise inserting the endoscopic biopsy instrument through the rhinoscope into the nose of the patient, and the step of obtaining a tissue sample may comprise obtaining a tissue sample from the nose of the patient.

The different aspects or part of the aspects of the disclosed embodiments may all be combined in any possible way, unless clearly contradictory. Any method or any step of a method should be seen also as a description of the elements necessary to carry out said method.

### BRIEF DESCRIPTION OF DRAWINGS

The invention is now described, by way of example, with reference to the accompanying drawings, on which:

FIG. 1 shows an embodiment of the endoscopic biopsy instrument in an exploded elevated view.

FIG. 2 shows a detailed sectional view of the handle portion of the endoscopic biopsy instrument.

FIG. 3 is a flow chart of a method of using the endoscopic biopsy instrument.

FIG. 4A shows a side view of the proximal portion of the endoscopic biopsy instrument and a sectional view of the distal portion of the endoscopic biopsy instrument when a tissue sample is being obtained.

FIG. 4B shows a side view of the proximal portion of the endoscopic biopsy instrument and a sectional view of the distal portion of the endoscopic biopsy instrument when the tissue sample has been retracted into the sleeve of the distal portion.

### DETAILED DESCRIPTION

In the following, a detailed description of embodiments of the invention will be given with reference to the accompanying drawings. It will be appreciated that the drawings are for illustration only and are not in any way restricting the scope of the invention. Thus, any references to directions, such as “up” or “down”, are only referring to the directions shown in the figures.

An endoscopic biopsy instrument for obtaining a tissue sample from the body of the patient is provided. The endoscopic biopsy instrument comprises a proximal portion adapted to remain outside of the body of the patient, when the endoscopic biopsy instrument is in use, and a distal portion adapted to be inserted into the body of the patient when the endoscopic biopsy instrument is in use. The endoscopic biopsy instrument further comprises a sleeve extending from the proximal portion to the distal portion, which may be a rigid or flexible sleeve, and a motion transferring member extending from the proximal portion to the distal portion within the sleeve. The motion transferring member may be a rigid or flexible motion transferring member depending on the specific application of the endoscopic biopsy instrument.

The distal portion comprises a distal end of the motion transferring member adapted to be connected to a rotatable cutting tip being extendable from a distal end of the sleeve, and being configured to obtain a tissue sample from within the body of the patient using rotary motion. The motion transferring member is configured to transfer rotary and linear motion, i.e. the motion transferring member is configured to transfer motion for extracting/retracting and rotating the rotatable cutting tip.

The proximal portion of the endoscopic biopsy instrument comprises a handle portion connected to the sleeve. The handle portion comprises a rotary motion element connected to the motion transferring member for transferring rotary motion to the rotatable cutting tip, and a linear motion element connected to the cutting tip via the motion transferring member, for extending and retracting the cutting tip from the distal portion of the sleeve. The handle portion further comprises a lockable adjustment device for, in situ, adjusting and locking a maximum length that the rotatable cutting tip extends from the distal portion of the sleeve, when being extended and retracted by the linear motion element. The handle portion enables the adjustment to take place after the endoscopic biopsy instrument has been inserted into the body of the patient and thus enables the adjustment to be performed in the position in which the tissue of the patient is obtained.

In one embodiment, the sleeve (or a portion of the sleeve) is an elongated sleeve which is flexible, such that the endoscopic biopsy instrument can be inserted through a channel of a flexible endoscope. As the motion transferring member extends within the sleeve, the sleeve has a larger diameter than the motion transferring member. The fact that the sleeve and the motion transferring member have different diameters causes the length relationship between the sleeve and the motion transferring member to be altered as the endoscopic biopsy instrument is bent. If the maximum length that the rotatable cutting tip extends from the distal portion of the sleeve is adjusted prior to the endoscopic biopsy instrument being inserted into the body of the patient, this maximum length will, due to the differences in diameter, be altered as the endoscopic biopsy instrument is bent. Further to that, a flexible motion transferring member is somewhat extended when used, e.g. a motion transferring member comprised of a cable may stretch somewhat when used even though the cable is tightly twisted, as the cable is made up of a plurality of strands and thus has internal cavities. Extension of the flexible motion transferring member alters the relationship between the length of the motion transferring member and the sleeve. In some embodiments, the sleeve and the motion transferring member can be made from different materials which are prolonged differently by thermal changes (such as when the instrument is inserted into the body of the patient). Being able to adjust the maximum length that the rotatable cutting tip extends from the distal portion of the sleeve in situ is thus a significant improvement which increases the precision with which a tissue sample can be obtained.

Endoscope is to be understood as any instrument for viewing or inserting tools for any form of manipulation inside of the body of a patient, in any location. An endoscope could for example be a gastroscope, a colonoscope, a bronchoscope, a rhinoscope, an arthroscope or a trocar.

Biopsy is to be understood as any removal of bodily cells or tissues using an instrument.

Motion transferring member is to be understood as any means for transferring a motion from a first location to a second location positioned at a distance from the first location. A motion transferring member could for example comprise a shaft, a flexible shaft, a Bowden cable, a universal joint, a worm drive, a bevel gear or a combination thereof.

Rotatable cutting tip is to be understood as any means adapted for cutting by means of rotation. According to one embodiment, the rotatable cutting tip could comprise a drill having a screw-like configuration with sharpened threads for cutting the tissue of a patient. Alternatively, the screw-like configuration could be adapted to extract tissue which could be cut against a sleeve in which the screw-like drill is positioned. In alternative embodiments, the rotatable cutting tip could be a cutting tip which is sharp in its distal end, such as a trephine blade.

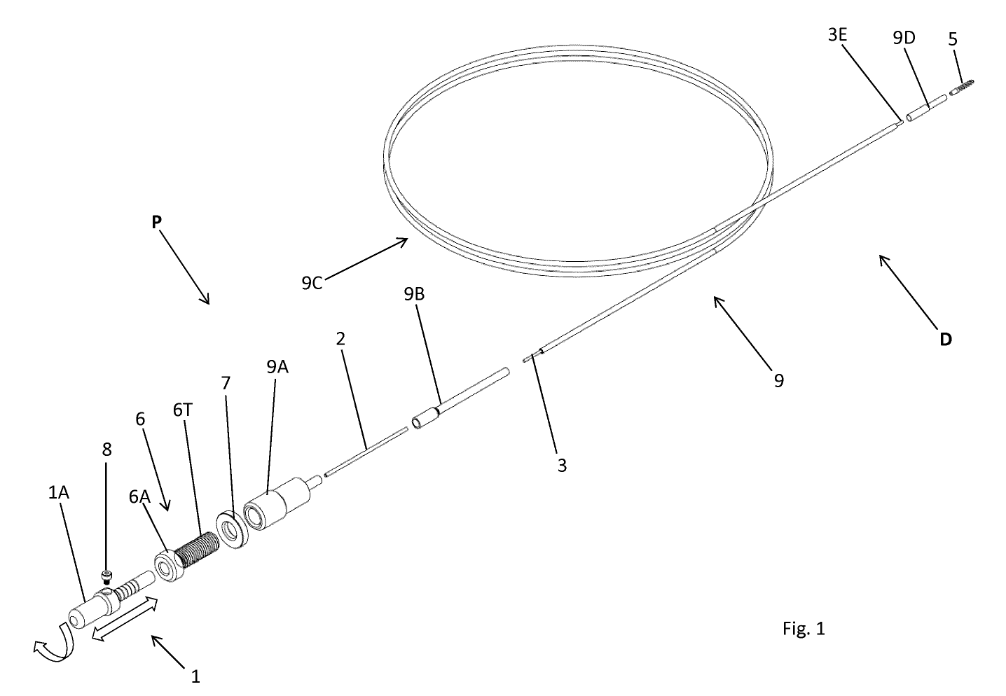

FIG. 1 shows an embodiment of the endoscopic biopsy instrument in an exploded elevated view. The endoscopic biopsy instrument comprises a proximal portion P adapted to remain outside of the body of the patient, when the endoscopic biopsy instrument is in use, and a distal portion D adapted to be inserted into the body of the patient when the endoscopic biopsy instrument is in use.

Starting from the proximal end of the endoscopic biopsy instrument, the instrument comprises a combined element 1 which is an element for manually generating rotary motion and linear motion. The combined element 1 enables the operator to single-handedly operate the biopsy instrument to cut and/or extract tissue using the rotary motion and cut or/and extract the tissue using the linear motion. However, it is equally conceivable that the rotary motion element 1 and the linear motion element 1 are separate elements in alternative embodiments.

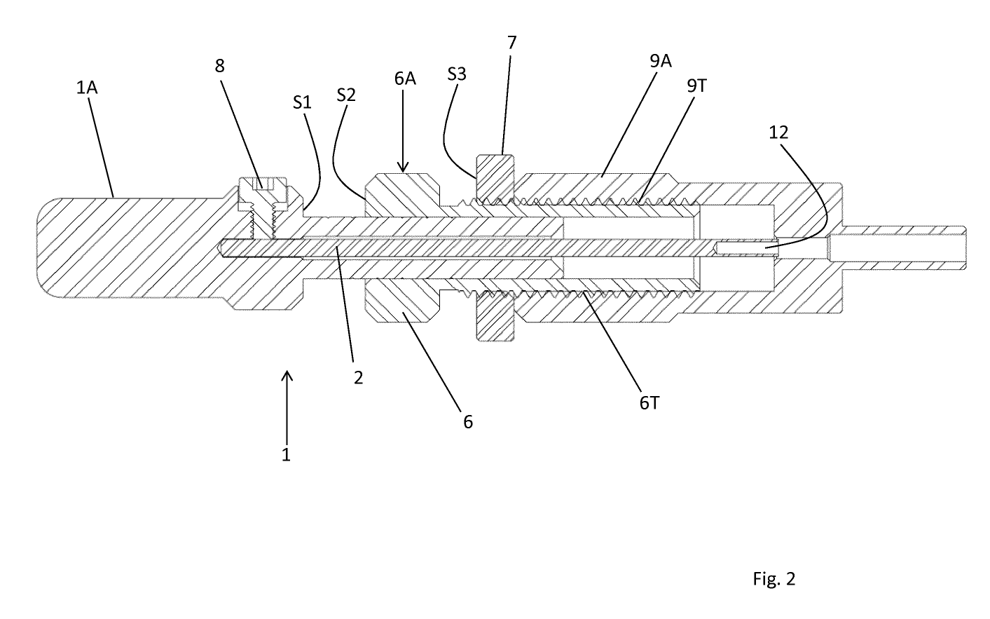

The combined element 1 is in the embodiment shown in FIG. 1 connected to a stiff shaft 2 extending from the combined element 1, through the adjustment sleeve 6 and the proximal portion 9A of the sleeve 9, which will be further described with reference to FIG. 2. The stiff shaft 2 is in turn connected to a flexible motion transferring member 3 being a flexible shaft 3 comprised of a stranded cable made from a plurality of strands being tightly twisted into a helix. The tightly twisted cable 3 is an important feature in endoscopic biopsy, as it enables the motions performed by the operator at the handle portion to be identically mirrored at the distal portion D of the endoscopic biopsy instrument.

The flexible shaft 3 is housed in a sleeve 9 comprising a first, short, rigid portion 9A having a larger diameter, a second rigid portion 9B, having a smaller diameter, a third flexible portion 9C, and a fourth rigid distal portion 9D, through which the rotatable cutting tip 5 extends. The flexible portion 9C of the sleeve 9 could for example comprise a helically wound metal sleeve, a flexible sleeve made from a polymer material, a woven flexible metal sleeve, or a combination thereof.

The distal end 3E of the motion transferring member 3 is fixated to the rotatable cutting tip 5. In the embodiment of FIG. 1, the rotatable cutting tip 5 is a screw-like drill with sharpened threads which drills into, and cuts, bodily matter inside the body of the patient. The fitting between the flexible shaft 3 and the rotatable cutting tip 5 could for example be a press fitting, a threaded fitting or a fitting created by means of material bonding.

Turning again to the proximal portion P of the endoscopic biopsy instrument, the proximal portion P comprises a handle portion which comprises (in addition to the combined element 1) a lockable adjustment device 6, 7, 9A for, in situ, adjusting and locking a maximum length that the rotatable cutting tip 5 extends from the distal portion 9D of the sleeve 9, when being extended and retracted by the combined element 1. The combined element 1 and the stiff shaft 2 extend within the lockable adjustment device 6, 7, 9A, which is further described with reference to FIG. 2.

The lockable adjustment device 6, 7, 9A shown in FIG. 1 comprises an adjustment sleeve 6, being axially displaceable in relation to the proximal portion 9A of the sleeve 9. The proximal portion 9A of the sleeve 9 comprises an internally threaded portion (shown in section in FIG. 2) comprising threads (9T of FIG. 2) being adapted to engage the threads 6T of the adjustment sleeve 6, such that the adjustment sleeve 6 can be axially displaced in relation to the sleeve 9, by the adjustment sleeve 6 and the sleeve 9 being radially rotated in relation to each other. The lockable adjustment device 6, 7, 9A further comprises a locking device 7 in the form of a threaded locking member 7 adapted to engage the threads 6T of the adjustment sleeve 6, and being adapted to be screwed fixedly against the proximal portion 9A, for axially locking the adjustment sleeve 6 in relation to the sleeve 9A and thereby adjusting and locking the maximum length that the rotatable cutting tip 5 extends from the distal portion 9D of the sleeve 9, when being extended and retracted by the linear motion element 1.

In alternative embodiments, the proximal portion of sleeve 9A comprises outer threads engaging inner threads of the adjustment sleeve and the locking device, such that the adjustment device and the locking member are axially displaced on the outside of the proximal portion 9A of the sleeve 9.

At least one of the combined element 1 and the lockable adjustment device 6, 7, 9A may comprise grip improving protrusions or recesses (not shown) which may comprise axially extending grooves in particular improving the operators grip when generating rotary motion.

The endoscopic biopsy instrument could for example be used in a channel of an endoscope being a gastroscope or a colonoscope, for obtaining a tissue sample from the digestive system of a patient, or a bronchoscope for obtaining a tissue sample from the airways of a patient, and may have a length exceeding one of 10 cm, 20 cm, 30 cm, 40 cm, 50 cm, 60 cm, 70 cm, 80 cm, 90 cm or 100 cm for being able to reach areas of the patient's digestive system or airways.

FIG. 2 shows a detailed sectional view of the handle portion of the proximal portion P of the embodiment of the endoscopic biopsy instrument of FIG. 1. From the left side of the FIG. 2, the combined element 1 is shown, comprising a manipulation portion 1A adapted to be manipulated by the operator. The combined element 1 comprises an internally threaded bore in which a set screw 8 is positioned. The set screw 8 engages a recess of the stiff shaft 2 and thereby axially locks the stiff shaft 2 in the combined element 1. The distal end of the stiff shaft 2 comprises an axially extending bore 12 adapted to receive the proximal end of the flexible shaft 3. The flexible shaft 3 is adapted to be fixated in the axially extending bore 12 by means of press fitting. The combined element 1 runs freely inside of the adjustment sleeve 6, and the proximal portion of the sleeve 9A and linear and rotary motion of the combined element 1, generated by the operator, thus causes extension, retraction and rotation of the rotatable cutting tip 5 at the distal portion D.

In the embodiment shown in FIGS. 1 and 2, the endoscopic biopsy instrument is configured for stepless operation of the extension and retraction of the rotatable cutting tip (5 of FIG. 1), and of the rotary motion of the cutting tip. Stepless operation makes it easier to obtain a grip in a hard tumor or bone, as the rotatable cutting tip can be rotated without linear displacement. Stepless operation also enables obtained tissue to be further transported along the threads of the rotatable cutting tip and further into the distal portion of the sleeve (9D of FIG. 1) and enables the rotatable cutting tip to be rotated at the same time as it is retracted into the sleeve. The endoscopic biopsy instrument can also be used to pass through tissue at a first area to obtain tissue at a second area, or to obtain tissue from a central location of a larger tissue portion.

The lockable adjustment device 6, 7, 9A will now be further described. The lockable adjustment device 6, 7, 9A of the embodiment of FIGS. 1 and 2 comprises an adjustment sleeve 6 comprising a manipulation portion 6A, adapted to be rotated by the operator, and a treaded portion 6T, engaging internal threads 9T of the proximal portion of the sleeve 9A, such that rotation of the adjustment sleeve 6 causes axial displacement of the adjustment sleeve in relation to the sleeve 9. The lockable adjustment device 6, 7, 9A further comprises a locking device 7 for locking the lockable adjustment device. The locking device 7 is adapted to be rotated by the operator and comprises internal threads 7T engaging the threads 6T of the adjustment sleeve 6, such that rotation of the locking device 7 causes axial displacement of the locking device 7 along the threaded portion 6T of the adjustment sleeve 6. When the locking device 7 is screwed tightly against the surface S3 of the proximal end of the sleeve 9, the locking ring 7 locks the adjustment sleeve 9 in relation to the sleeve 9 and thus locks the maximum length that the rotatable cutting tip (5 of FIG. 1) extends from the distal portion (9D of FIG. 1) of the sleeve 9, when the rotatable cutting tip is extended and retracted by the combined element 1.

The combined element shown in FIG. 1 has an innermost position, in which the surface S1 of the manipulation portion 1A of the combined element 1 engages the distal surface S2 of the adjustment sleeve 6. When the combined element is in its innermost position, the cutting tip extends maximally from the distal end of the sleeve. The adjustment of the lockable adjustment device adjusts the distance between the proximal end of the sleeve 9 and the innermost position and thus adjusts the maximum length that the rotatable cutting tip (5 of FIG. 1) extends from the distal portion (9D of FIG. 1), when being extended and retracted by the combined element 1.

In the embodiment shown in FIGS. 1 and 2, the lockable adjustment device 6, 7, 9A can steplessly adjust the maximum length that the rotatable cutting tip extends from the sleeve, however, in alternative embodiments, the adjustment could be performed in steps.

In alternative embodiments, the lockable adjustment device may comprise a locking device comprising a radially displaceable part adapted to engage at least one of: the motion transferring member, the sleeve and the adjustment sleeve, for locking the maximum length that the rotatable cutting tip extends from the sleeve. The radially displaceable part could lock against the motion transferring member, the sleeve, or the adjustment sleeve by means of friction, or by engaging recesses of the sleeve or adjustment sleeve. The radially displaceable part could be a spring loaded part similar to the spring loaded part of a cord lock for apparel. The radially displaceable part may be adapted to release when pressed, such that the operator can press the radially displaceable part, adjust the a the lockable adjustment device, and release the radially displaceable part for locking the lockable adjustment device.

In a further alternative configuration, the radially displaceable part comprises a silicone disc having a single straight slit or a Y or + shaped slit. When the silicone disc is radially compressed, the slit is opened which enables the motion transferring member, the sleeve or the adjustment sleeve to be displaced in relation to the silicone disc. When the radial pressure is released, the edges of the slit engage the motion transferring member, the sleeve or the adjustment sleeve, locking the lockable adjustment device.

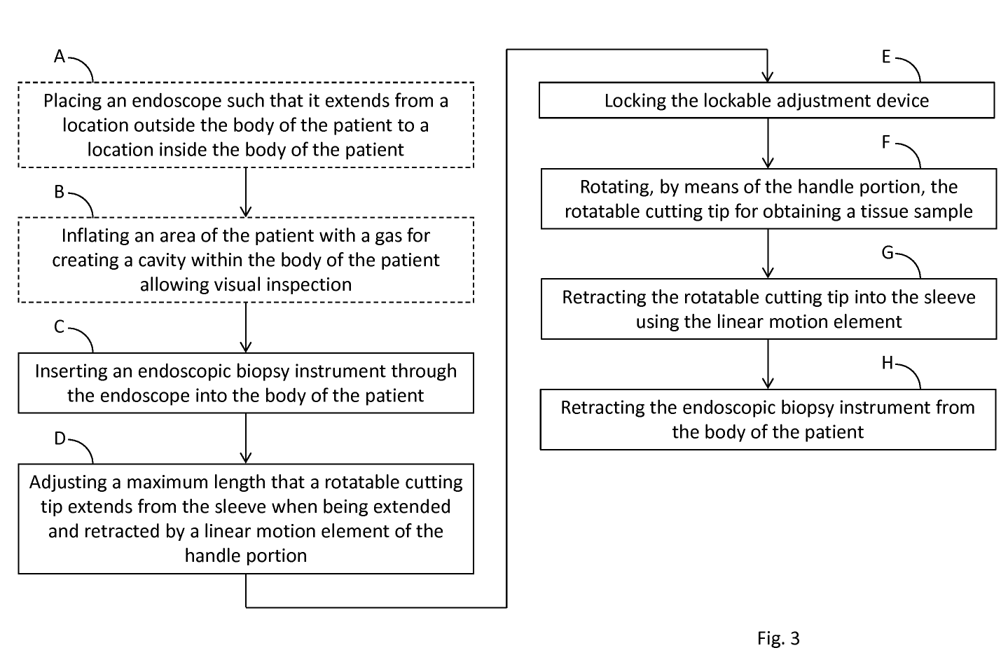

FIG. 3 is a flow chart of a method of using the endoscopic biopsy instrument of FIGS. 1 and 2 for obtaining a tissue sample from the body of a patient. The method comprises the steps of C inserting the endoscopic biopsy instrument through an endoscope into the body of the patient, D adjusting a maximum length that the rotatable cutting tip extends from the sleeve when being extended and retracted by a linear motion element of the handle portion, using a lockable adjustment device of the endoscopic biopsy instrument, E locking the lockable adjustment device, F rotating, by means of the handle portion, the rotatable cutting tip for obtaining a tissue sample, G retracting the rotatable cutting tip into the sleeve using the linear motion element, and H retracting the endoscopic biopsy instrument from the body of the patient.

The method may be preceded by the steps of A placing the endoscope such that it extends from a location outside the body of the patient to a location inside the body of the patient and B inflating an area of the patient with a gas for creating a cavity within the body of the patient allowing visual inspection.

The endoscope may comprise a camera for visual inspection, such that the step of D adjusting a maximum length that the rotatable cutting tip extends from the sleeve can be performed under visual inspection.

The endoscope may be a gastroscope, and the step of C inserting the endoscopic biopsy instrument may comprise inserting the endoscopic biopsy instrument through the gastroscope into the digestive system of the patient, and the step of F obtaining a tissue sample may comprise obtaining a tissue sample from the digestive system of the patient.

Alternatively, the endoscope may be a colonoscope, and the step of C inserting the endoscopic biopsy instrument through an endoscope into the body of the patient may comprise inserting the endoscopic biopsy instrument through the colonoscope into the digestive system of the patient, and the step of F obtaining a tissue sample comprises obtaining a tissue sample from the digestive system of the patient.

Alternatively, the endoscope may be a bronchoscope, and the step of C inserting the endoscopic biopsy instrument through an endoscope into the body of the patient may comprise inserting the endoscopic biopsy instrument through the bronchoscope into the airways of the patient, and the step of F obtaining a tissue sample may comprise obtaining a tissue sample from the airways of the patient.

Alternatively, the endoscope may be a rhinoscope, and the step of C inserting the endoscopic biopsy instrument through an endoscope into the body of the patient may comprise inserting the endoscopic biopsy instrument through the rhinoscope into the nose of the patient, and the step of F obtaining a tissue sample may comprise obtaining a tissue sample from the nose of the patient.

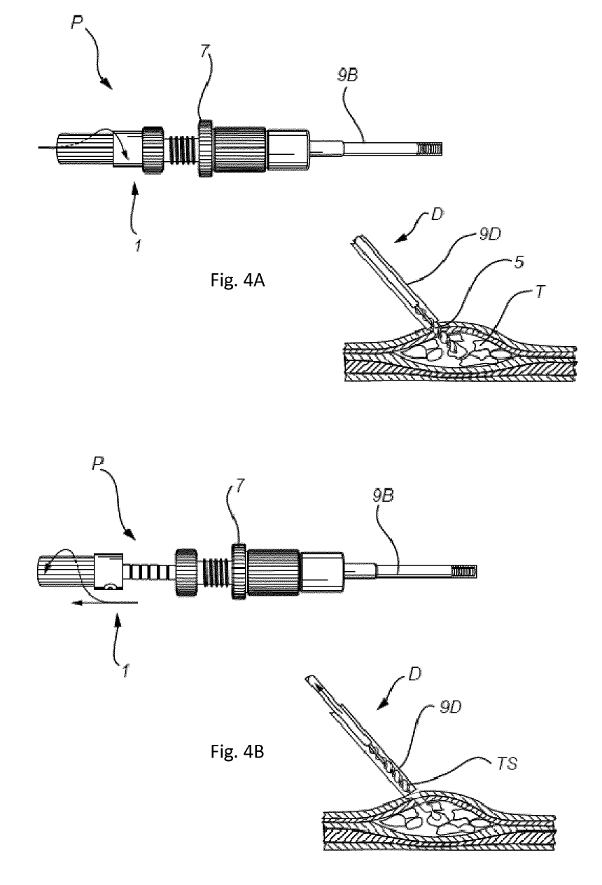

FIG. 4A shows a side view of the proximal portion P of the endoscopic biopsy instrument and a sectional view of the distal portion D of the endoscopic biopsy instrument when a tissue sample (TS in FIG. 4B) is being obtained. The distal portion D has been inserted into the body of the patient through for example an endoscopic biopsy instrument. By means of the combined element 1 and the locking device 7 (such as explained in further detail with reference to FIG. 2), the maximum length that the rotatable cutting tip 5 extends from the distal portion of the sleeve 9D is being set in situ. The tissue sample (TS of FIG. 4B) is being collected by the rotation of the combined element 1, causing the cutting tip 5 to rotate and “drill” into the tissue T of the patient until the maximum extension from the sleeve is reached.

FIG. 4B shows the same view of the endoscopic biopsy instrument as in FIG. 4A but when the tissue sample TS is being retracted into the distal portion of the sleeve 9D. The combined element 1 is being simultaneously retracted and rotated clockwise, such that the cutting tip 5 continuously exerts a retracting force on the tissue sample TS, such that the obtained tissue sample TS is severed from the rest of the tissue and remains in the distal part of the sleeve 9D as the distal portion D of the endoscopic biopsy instrument is being retracted from the body of the patient.

The different aspects or part of the aspects of the disclosed embodiments may all be combined in any possible way, unless clearly contradictory. Any method or any step of a method should be seen also as a description of the elements necessary to carry out said method. Any detailed description should be interpreted in its broadest outline as a general description of the inventive idea.

Numbered Examples

In the following, numbered exemplifying instruments are provided. The numbered exemplifying instruments are not to be seen as limiting the scope of the invention, which is defined by the appended claims.

1. An exemplifying endoscopic biopsy instrument for obtaining a tissue sample from a body of a patient, the endoscopic biopsy instrument comprising:
a proximal portion (P) adapted to remain outside of the body of the patient in use, and
a distal portion (D) adapted to be inserted into the body of the patient in use,
a sleeve (9) extending from the proximal portion to the distal portion, and
a motion transferring member (3) extending from the proximal portion to the distal portion within the sleeve, the motion transferring member being configured to transfer rotary and linear motion,
   wherein the distal portion comprises a distal end (3E) of the motion transferring member adapted to be connected to a rotatable cutting tip (5) being extendable from a distal end of the sleeve, and being configured to obtain a tissue sample from within the body of the patient using rotary motion, and
   wherein the proximal portion comprises a handle portion connected to the sleeve, the handle portion comprising:
a rotary motion element (1) connected to the motion transferring member for transferring rotary motion to the rotatable cutting tip,
a linear motion element (1) connected to the cutting tip via the motion transferring member, for extending and retracting the cutting tip from the distal portion of the sleeve, and
a lockable adjustment device (6, 7, 9A) for, in situ, adjusting and locking a maximum length that the rotatable cutting tip extends from the distal portion of the sleeve, when being extended and retracted by the linear motion element.

2. The exemplifying endoscopic biopsy instrument according to claim 1, wherein the linear motion element has an innermost position, in which the cutting tip extends maximally from the distal end of the sleeve, and wherein the lockable adjustment device is adapted to adjust the distance between a proximal end of the sleeve and the innermost position.

3. The exemplifying endoscopic biopsy instrument according to any one of the preceding claims, wherein the lockable adjustment device comprises an adjustment sleeve (6) being axially displaceable in relation to the sleeve, and wherein the lockable adjustment device further comprises a locking device (7) for axially locking the adjustment sleeve in relation to the sleeve and thereby adjusting and locking the maximum length that the rotatable cutting tip extends from the sleeve, when being extended and retracted by the linear motion element.

4. The exemplifying endoscopic biopsy instrument according to claim 3, wherein a proximal portion of the sleeve comprises a threaded portion (9T), and wherein the adjustment sleeve comprises threads (6T) being adapted to engage the threads of the sleeve, such that the adjustment sleeve can be axially displaced in relation to the sleeve by the adjustment sleeve and the sleeve being radially rotated in relation to each other.

5. The exemplifying endoscopic biopsy instrument according to claim 4, wherein the locking device comprises a threaded locking member (7) adapted to engage the threads of at least one of the sleeve and the adjustment sleeve, for locking the sleeve in relation to the adjustment sleeve.

6. The exemplifying endoscopic biopsy instrument according to any one of the preceding claims, wherein the lockable adjustment device comprises a locking device comprising a radially displaceable part adapted to engage at least one of the motion transferring member, the sleeve and the adjustment sleeve, for locking the maximum length that the rotatable cutting tip extends from the sleeve, when being extended and retracted by the linear motion element.

7. The exemplifying endoscopic biopsy instrument according to any one of the preceding claims, wherein the rotary motion element and the linear motion element is a combined element (1), such that rotary and linear motion can be generated single-handedly.

8. The exemplifying endoscopic biopsy instrument according to any one of the preceding claims, wherein the endoscopic biopsy instrument is configured for stepless operation of at least one of:
the adjustment of the maximum length that the rotatable cutting tip extends from the sleeve,
the extension and retraction of the rotatable cutting tip, and
the rotary motion of the cutting tip.

9. The exemplifying endoscopic biopsy instrument according to any one of the preceding claims, wherein at least one of:
the sleeve is a flexible sleeve (9C), and
the motion transferring member is a flexible motion transferring member (3).

10. The exemplifying endoscopic biopsy instrument according to any one of the preceding claims, comprising the rotatable cutting tip.

11. The exemplifying endoscopic biopsy instrument according to claim 10, wherein the rotatable cutting tip comprises a drill (5).

12. The exemplifying endoscopic biopsy instrument according to claim 9, wherein the flexible motion transferring member is a flexible shaft (3).

13. The exemplifying endoscopic biopsy instrument according to any one of the preceding claims, wherein the motion transferring member has a length exceeding 30 cm.

14. The exemplifying endoscopic biopsy instrument according to any one of the preceding claims, wherein the endoscopic biopsy instrument is configured for obtaining a tissue sample in the digestive system of the patient.

15. The exemplifying endoscopic biopsy instrument according to claim 9, wherein the flexible sleeve comprises at least one of:
a helically wound metal sleeve,
flexible sleeve made from a polymer material, and
a woven flexible metal sleeve.

16. The exemplifying endoscopic biopsy instrument according to claim 3, wherein the linear motion element extends partially inside of the adjustment sleeve and the adjustment sleeve extends partially inside of the sleeve.

17. The exemplifying endoscopic biopsy instrument according to any one of the preceding claims, wherein at least one of: the rotary motion element, the linear motion element and the lockable adjustment device comprises grip improving protrusions or recesses.

18. The exemplifying endoscopic biopsy instrument according to claim 17, wherein the grip improving protrusions or recesses comprises axially extending grooves.

19. The exemplifying endoscopic biopsy instrument according to claims 6, wherein the adjustment sleeve is a compressible adjustment sleeve comprising the radially displaceable part, and wherein:
compression of the compressible adjustment sleeve disengages the radially displaceable part from the motion transferring member the sleeve or the adjustment sleeve, such that the maximum length that the rotatable cutting tip extends from the distal portion of the sleeve can be adjusted, and
the release of the compression of the compressible adjustment sleeve engages the radially displaceable part to the motion transferring member the sleeve or the adjustment sleeve, such that the lockable adjustment device is locked.

20. The exemplifying endoscopic biopsy instrument according to claim 19, wherein at least one of the radially displaceable part and the compressible adjustment sleeve comprises a polymer material.

Numbered Examples

In the following, numbered exemplifying methods are provided. The numbered exemplifying methods are not to be seen as limiting the scope of the invention, which is defined by the appended claims.

1. An exemplifying method of obtaining a tissue sample from a body of a patient using an endoscopic biopsy instrument comprising a sleeve and a rotatable cutting tip operated from a handle portion of the endoscopic biopsy instrument, the cutting tip being configured to obtain a tissue sample from within the body of the patient using rotary motion, the method comprising:
inserting the endoscopic biopsy instrument through an endoscope into the body of the patient,
adjusting a maximum length that the rotatable cutting tip extends from the sleeve when being extended and retracted by a linear motion element of the handle portion, using a lockable adjustment device of the endoscopic biopsy instrument,
locking the lockable adjustment device,
rotating, by means of the handle portion, the rotatable cutting tip for obtaining a tissue sample,
retracting the rotatable cutting tip into the sleeve using the linear motion element, and
retracting the endoscopic biopsy instrument from the body of the patient.

2. The method according to exemplifying method 1, wherein the method is preceded by the step of placing the endoscope such that it extends from a location outside the body of the patient to a location inside the body of the patient.

3. The method according to exemplifying method 2, wherein the endoscope comprises a camera for visual inspection and wherein the step of adjusting a maximum length that the rotatable cutting tip extends from the sleeve is performed under visual inspection.

4. The method according to exemplifying method 2, wherein the endoscope is a gastroscope, and wherein the step of inserting the endoscopic biopsy instrument through an endoscope into the body of the patient comprises inserting the endoscopic biopsy instrument through the gastroscope into the digestive system of the patient, and wherein the step of obtaining a tissue sample comprises obtaining a tissue sample for the digestive system of the patient.

5. The method according to exemplifying method 2, wherein the endoscope is a colonoscope, and wherein the step of inserting the endoscopic biopsy instrument through an endoscope into the body of the patient comprises inserting the endoscopic biopsy instrument through the colonoscope into the digestive system of the patient, and wherein the step of obtaining a tissue sample comprises obtaining a tissue sample for the digestive system of the patient.

6. The method according to exemplifying method 2, wherein the endoscope is a bronchoscope, and wherein the step of inserting the endoscopic biopsy instrument through an endoscope into the body of the patient comprises inserting the endoscopic biopsy instrument through the bronchoscope into the airways of the patient, and wherein the step of obtaining a tissue sample comprises obtaining a tissue sample for the airways of the patient.

7. The method according to exemplifying method 2, wherein the endoscope is a rhinoscope, and wherein the step of inserting the endoscopic biopsy instrument through an endoscope into the body of the patient comprises inserting the endoscopic biopsy instrument through the rhinoscope into the nose of the patient, and wherein the step of obtaining a tissue sample comprises obtaining a tissue sample for the nose of the patient.

8. The method according to exemplifying method 1, further comprising the step of inflating an area of the patient with a gas for creating a cavity within the body of the patient allowing visual inspection.

1-20. (canceled)

21. An endoscopic biopsy instrument for obtaining a tissue sample from a body of a patient, the endoscopic biopsy instrument comprising:
a proximal portion configured to remain outside of the body of the patient in use;
a distal portion configured to be inserted into the body of the patient in use;
a sleeve extending from the proximal portion to the distal portion; and
a motion transferring member positioned within the sleeve, the motion transferring member extending from the proximal portion to the distal portion and being configured to transfer rotary motion and linear motion,
wherein the distal portion comprises a distal end of the motion transferring member,
wherein the distal end of the motion transferring member is configured to be connected to a rotatable cutting tip being extendable from a distal end of the sleeve, and the rotatable cutting tip being configured to obtain a tissue sample from within the body of the patient using the rotary motion, and
wherein the proximal portion comprises a handle portion connected to the sleeve, the handle portion comprising:
a rotary motion element coupled to the motion transferring member, wherein the rotary motion element is configured to transfer the rotary motion to the rotatable cutting tip;
a linear motion element coupled to the motion transferring member, wherein the linear motion element is configured to extend and retract the rotatable cutting tip from the distal portion of the sleeve; and
a lockable adjustment device configured to adjust and lock the rotatable cutting tip a maximum length that the rotatable cutting tip extends from the distal portion of the sleeve when the rotatable cutting tip is extended and retracted by the linear motion element and the rotatable cutting tip is rotated by the rotary motion element.

22. The endoscopic biopsy instrument of claim 1, wherein the linear motion element comprises an innermost position in which the rotatable cutting tip maximally extends from the distal end of the sleeve, and wherein the lockable adjustment device is configured to adjust the distance between a proximal end of the sleeve and the innermost position of the linear motion element.

23. The endoscopic biopsy instrument of claim 1, wherein the lockable adjustment device comprises an adjustment sleeve being axially displaceable in relation to the sleeve, and wherein the lockable adjustment device comprises a locking device for axially locking the adjustment sleeve in relation to the sleeve and adjusting and locking the maximum length that the rotatable cutting tip extends from the sleeve when the rotatable cutting tip extends and retracts by the linear motion element.

24. The endoscopic biopsy instrument of claim 3, wherein a proximal portion of the sleeve comprises a threaded portion, and wherein the adjustment sleeve comprises threads being adapted to engage the threaded portion of the sleeve such that the adjustment sleeve is axially displaced in relation to the sleeve by the adjustment sleeve and the sleeve being radially rotated in relation to each other.

25. The endoscopic biopsy instrument of claim 4, wherein the locking device comprises a threaded locking member configured to engage at least one of the threaded portion of the sleeve and the threads of the adjustment sleeve for locking the sleeve in relation to the adjustment sleeve.

26. The endoscopic biopsy instrument of claim 1, wherein the lockable adjustment device comprises a locking device comprising a radially displaceable part adapted to engage at least one of the motion transferring member, the sleeve, and the adjustment sleeve, wherein the locking device is configured to lock the rotatable cutting tip the maximum length that the rotatable cutting tip extends from the sleeve when the rotatable cutting tip extends and retracts by the linear motion element.

27. The endoscopic biopsy instrument of claim 1, wherein the rotary motion element and the linear motion element are a combined element such that the rotary motion and the linear motion are generated single-handedly.

28. The endoscopic biopsy instrument of claim 1, wherein the endoscopic biopsy instrument is configured for stepless operation of at least one of the adjustment of the maximum length that the rotatable cutting tip extends from the sleeve, the extension and retraction of the rotatable cutting tip, or the rotary motion of the rotatable cutting tip.

29. The endoscopic biopsy instrument of claim 1, wherein the sleeve is a flexible sleeve or the motion transferring member is a flexible motion transferring member.

30. The endoscopic biopsy instrument of claim 9, wherein the flexible motion transferring member is a flexible shaft.

31. The endoscopic biopsy instrument of claim 1, wherein the motion transferring member is longer than 30 cm.

32. The endoscopic biopsy instrument of claim 3, wherein the linear motion element extends partially inside of the adjustment sleeve and the adjustment sleeve extends partially inside of the sleeve.

33. The endoscopic biopsy instrument of claims 6, wherein the adjustment sleeve is a compressible adjustment sleeve comprising the radially displaceable part, and wherein:
compression of the compressible adjustment sleeve disengages the radially displaceable part from the motion transferring member of the sleeve or the adjustment sleeve such that the maximum length that the rotatable cutting tip extends from the distal portion of the sleeve is adjustable; and
release of the compression of the compressible adjustment sleeve engages the radially displaceable part to the motion transferring member of the sleeve or the adjustment sleeve such that the lockable adjustment device is locked.

34. The endoscopic biopsy instrument of claim 13, wherein at least one of the radially displaceable part and the compressible adjustment sleeve comprises a polymer material.

35. A method of obtaining a tissue sample from a body of a patient using an endoscopic biopsy instrument comprising a sleeve and a rotatable cutting tip operated from a handle portion of the endoscopic biopsy instrument, the cutting tip being configured to obtain a tissue sample from within the body of the patient using rotary motion, the method comprising:
inserting the endoscopic biopsy instrument through an endoscope into the body of the patient;
using a lockable adjustment device of the endoscopic biopsy instrument, adjusting the rotatable cutting tip a maximum length that the rotatable cutting tip extends from the sleeve when being extended and retracted by a linear motion element of the handle portion;
locking the lockable adjustment device;
rotating, via the handle portion, the rotatable cutting tip for obtaining a tissue sample;
retracting the rotatable cutting tip into the sleeve using the linear motion element; and
retracting the endoscopic biopsy instrument from the body of the patient.

36. The method of claim 15, further comprising placing the endoscope such that it extends from a location outside the body of the patient to a location inside the body of the patient.

37. The method of claim 15, wherein the endoscope is a gastroscope, and wherein inserting the endoscopic biopsy instrument through an endoscope into the body of the patient comprises inserting the endoscopic biopsy instrument through the gastroscope into the digestive system of the patient, and wherein obtaining a tissue sample comprises obtaining a tissue sample for the digestive system of the patient.

38. The method of claim 15, wherein the endoscope is a colonoscope, and wherein inserting the endoscopic biopsy instrument through the endoscope into the body of the patient comprises inserting the endoscopic biopsy instrument through the colonoscope into the digestive system of the patient, and wherein obtaining a tissue sample comprises obtaining a tissue sample for the digestive system of the patient.

39. The method of claim 15, wherein the endoscope is a bronchoscope, and wherein inserting the endoscopic biopsy instrument through the endoscope into the body of the patient comprises inserting the endoscopic biopsy instrument through the bronchoscope into the airways of the patient, and wherein obtaining a tissue sample comprises obtaining a tissue sample for the airways of the patient.

40. The method of claim 15, wherein the endoscope is a rhinoscope, and wherein inserting the endoscopic biopsy instrument through the endoscope into the body of the patient comprises inserting the endoscopic biopsy instrument through the rhinoscope into the nose of the patient, and wherein obtaining a tissue sample comprises obtaining a tissue sample for the nose of the patient.
